(19) United States
(12) Patent Application Publication
(10) Pub. No.: US 2023/0009905 A1
(43) Pub. Date: Jan. 12, 2023

(54) APPARATUS AND METHOD FOR ESTIMATING BIOLOGICAL INFORMATION

(71) Applicant: SAMSUNG ELECTRONICS CO.,LTD., Suwon-si (KR)

(72) Inventors: Ka Ram CHOI, Yongin-si (KR); So Young Lee, Suwon-si (KR); Ho Jun Chang, Seoul (KR); Yun S Park, Suwon-si (KR); Hyeong Seok Jang, Seoul (KR)

(73) Assignee: SAMSUNG ELECTRONICS CO., LTD, Suwon-si (KR)

(21) Appl. No.: 17/724,824

(22) Filed: Apr. 20, 2022

(30) Foreign Application Priority Data
Jul. 8, 2021 (KR) 10-2021-0089934

(51) Int. Cl. A61B 5/145; A61B 5/1455; A61B 5/00; G01N 21/359

(52) U.S. Cl. CPC A61B 5/14532; A61B 5/1455; A61B 5/0075; A61B 5/14546; G01N 21/359; A61B 2560/0223

(57) ABSTRACT

An apparatus for estimating blood glucose is provided. The apparatus for estimating blood glucose may include a spectrometer configured to measure a spectrum from an object, and a processor configured to monitor a change in a contact state between the object and the spectrometer, determine a calibration section based on the monitored change in the contact state, extract a background signal from a spectrum of the determined calibration section, and generate a personalized blood glucose estimation model based on the extracted background signal.

### CROSS-REFERENCE TO RELATED APPLICATION(S)

This application claims the benefit under 35 USC § 119(a) of Korean Patent Application No. 10-2021-0089934, filed on Jul. 8, 2021, in the Korean Intellectual Property Office, the disclosure of which is incorporated herein by reference in its entirety.

### BACKGROUND

### 1. Field

Apparatuses and methods consistent with example embodiments relate to non-invasively obtaining biological information, such as blood glucose.

### 2. Description of Related Art

Diabetes is a chronic disease that causes various complications and can be hardly cured, and hence people with diabetes are advised to check their blood glucose regularly to prevent complications. In particular, when insulin is administered to control blood glucose, the blood glucose levels may have to be closely monitored to avoid hypoglycemia and control insulin dosage. There are several ways to monitor glucose levels, from invasive finger pricking testing to non-invasive glucose monitoring without causing pain. The invasive method may provide high reliability in measurement, but may cause pain and inconvenience as well as an increased risk of disease infections due to the use of injection. Recently, extensive research has been conducted on non-invasive measurements of body components, such as blood glucose, based on spectrum analysis without collecting blood.

### SUMMARY

According to an aspect of the present application, an apparatus for estimating blood glucose, may include: a spectrometer configured to measure a spectrum from an object; and a processor configured to monitor a change in a contact state between the object and the spectrometer, determine a calibration section based on the monitored change in the contact state, extract a background signal from the spectrum of the determined calibration section, and generate a personalized blood glucose estimation model based on the extracted background signal.

The spectrometer may include a light source configured to emit light to the object and a detector configured to detect a light signal by receiving the light scattered or reflected from the object.

The apparatus may further include: at least one of a force sensor, an impedance sensor, a motion sensor, or a gyro sensor, wherein the processor may be further configured to monitor the change in the contact state between the object and the spectrometer based on measurement data of the sensor.

The processor may be further configured to compare a reference spectrum in a stable state with the spectrum measured by the spectrometer to monitor the change in the contact state between the object and the spectrometer.

The processor may be further configured to determine that a section in which the monitored change in the contact state is greater than or equal to a predetermined reference is the calibration section.

The processor may be further configured to induce the change in the contact state when a calibration request is received, or when there is no section in which the change in the contact state is greater than or equal to a predetermined reference within a predetermined time from a start of a spectrum measurement, or during an entire time of the spectrum measurement.

The processor may be further configured to induce the change in the contact state by controlling a movement of the spectrometer.

The apparatus may further include a display configured to display a graphic object guiding the object to move in a predetermined direction.

The processor may be further configured to extract the background signal, based on the spectrum of the determined calibration section, by using at least one of differential, principal component analysis, independent component analysis, non-negative matrix factorization, or auto-encoding.

The processor may be further configured to compare the extracted background signal with at least one of a reference signal or a user's existing background signal present in a user-specific personalized background signal database to calculate a similarity or correlation therebetween, and verify whether the extracted background signal is valid, based on the calculated similarity or correlation.

When the extracted background signal is determined to be invalid, the processor may be further configured to re-determine a calibration section from the measured spectrum, extract another background signal from the spectrum of the determined calibration section, or guide the spectrometer to obtain another spectrum.

The processor may be further configured to perform pre-processing including at least one of baseline correction, scattering correction, normalization, or filtering on the extracted background signal.

The spectrometer may be further configured to measure the spectrum from the object at a time of estimating blood glucose and the processor is further configured to estimate the blood glucose based on the spectrum measured at the time of estimating blood glucose and the personalized blood glucose estimation model.

The apparatus may further include a storage configured to include a reference signal and a user-specific personalized background signal database, wherein the storage may be configured to store the extracted background signal in the personalized background signal database.

According to another aspect of the disclosure, there is provided a method of estimating blood glucose, including: measuring a spectrum from an object; monitoring a change in a contact state between the object and a spectrometer; determining a calibration section based on the monitored change in the contact state; extracting a background signal from the spectrum of the determined calibration section; and generating a personalized blood glucose estimation model based on the extracted background signal.

The determining of the calibration section may include determining that a section in which the monitored change in the contact state is greater than or equal to a predetermined reference is the calibration section.

The method may further include inducing the change in the contact state when a calibration request is received, or when there is no section in which the change in the contact state is greater than or equal to a predetermined reference within a predetermined time from a start of a spectrum measurement, or during an entire time of the spectrum measurement.

The method may include comparing the extracted background signal with at least one of a reference signal or a user's existing background signal present in a user-specific personalized background signal database to calculate a similarity or correlation therebetween, and verifying whether the extracted background signal is valid, based on the calculated similarity or correlation.

According to another aspect of the present disclosure, a wearable device may include: an optical sensor including a plurality of light emitting diodes (LEDs) configured to emit at least a green light, a red light, an infrared light to a user, and a plurality of photodiodes configured to measure light reflected back from the user; a motion sensor configured to obtain a motion signal by measuring a motion of the user while the wearable device is worn by the user; and a processor configured to: obtain personal information of the user, the personal information including information of a gender and an age of the user; during a calibration stage, control the optical sensor to obtain a reference biological signal while the user in a resting state and the wearable device is in contact with the user; and during an estimation stage, obtain a biological signal based on the light measured by the plurality of photodiode detectors, correct the biological signal based on the motion signal, the personal information, and the reference signal, and obtain health information of the user based on the corrected biological signal.

The processor may be further configured to: extract a background signal from the reference biological signal that is obtained during the calibration stage; and correct the biological signal based the background signal of the reference signal.

### BRIEF DESCRIPTION OF THE DRAWINGS

The above and/or other aspects will be more apparent by describing certain example embodiments, with reference to the accompanying drawings, in which:

FIG. 1 is a block diagram illustrating an apparatus for estimating a body component according to an exemplary embodiment;

FIG. 2 is a block diagram illustrating an apparatus for estimating blood glucose according to another exemplary embodiment;

FIG. 3 illustrates an example of a graphic object guiding an object to move in a predetermined direction;

FIG. 4 is a flowchart illustrating a method of estimating blood glucose according to an exemplary embodiment;

FIGS. 5A to 5C are flowcharts illustrating a method of estimating blood glucose according to another exemplary embodiment;

FIG. 6 is a flowchart illustrating a method of estimating blood glucose according to still another exemplary embodiment;

FIG. 7 illustrates a wearable device according to an exemplary embodiment; and

FIG. 8 illustrates a smart device according to an exemplary embodiment.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

It will be understood that, although the terms first, second, etc. may be used herein to describe various elements, these elements should not be limited by these terms. These terms are only used to distinguish one element from another. Also, the singular forms are intended to include the plural forms as well, unless the context clearly indicates otherwise. In the specification, unless explicitly described to the contrary, the word “comprise” and variations such as “comprises” or “comprising,” will be understood to imply the inclusion of stated elements but not the exclusion of any other elements. Terms such as “unit” and “module” denote units that process at least one function or operation, and they may be implemented by using hardware, software, or a combination of hardware and software.

Hereinafter, embodiments of an apparatus and method for estimating a body component will be described with reference to the drawings.

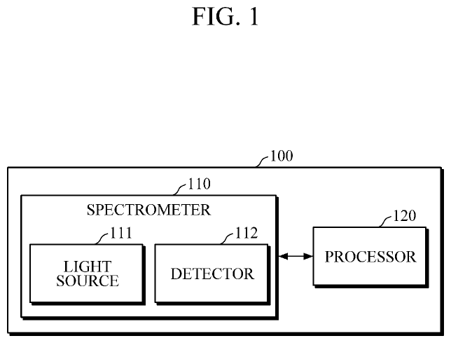

FIG. 1 is a block diagram illustrating an apparatus for estimating a body component according to an exemplary embodiment. An apparatus 100 for estimating a body component may be mounted in a wearable device that a user can wear. In particular, the wearable device may include various types of wearable devices, such as a wristwatch type, a bracelet type, a wristband type, a ring-type, an eyeglass type, a hair band type, and the like, and is not particularly limited in its shape or size.

Referring to FIG. 1, the apparatus 100 for estimating a health state and a body component includes a spectrometer 110 and a processor 120. The spectrometer 110 may be also referred to as an optical sensor.

In particular, the body component may include blood glucose, antioxidant, lactic acid, alcohol, cholesterol, triglyceride, and the like. Hereinafter, for convenience of explanation, a description will be given of blood glucose as the body component.

The spectrometer 110 may measure a spectrum from an object. In particular, the spectrometer 110 may use infrared spectroscopy or Raman spectroscopy, but is not limited thereto, such that a spectrum may be measured using various spectroscopic techniques. The object may be a skin of the user, for example, an upper area of the wrist through which capillary blood or venous blood passes, a region of a wrist surface adjacent to the radial artery, or a distal body portion, such as a finger, a toe, an earlobe, or the like, which has a high density of blood vessels. However, the examples of the object are not limited thereto.

The spectrometer 110 may include one or more light sources 111 and one or more detectors 112. The light source 111 may emit light to the object of the object, and the detector 112 may detect a light signal by receiving light scattered or reflected from the object.

The light source 111 may include a light emitting diode (LED), a laser diode, a phosphor, or the like, and may emit light in a near infrared ray (NIR) range or in a mid-infrared ray (MIR) range. In particular, the light source 111 may be configured as an array of a plurality of light sources configured to emit light in various wavelength ranges in order to obtain accurate spectral data. When the light source 111 emits light to the user's skin, which is the object, according to a control signal of the processor 120, the emitted light passes through the user's skin and reaches the body tissue, and the light that reaches the body tissue may be scattered or reflected by the body tissue and return through the user's skin.

The detector 112 may measure a spectrum by detecting the light returning through the user's skin. In particular, the detector 112 may include a photodiode, a phototransistor, or the like. However, the detector 112 is not limited thereto, and may include a complementary metal-oxide semiconductor (CMOS) image sensor, a charge-coupled device (CCD), an image sensor, and the like.

The plurality of light sources 111 may be configured to emit light of the same wavelength or light of different wavelengths. For example, the light source may emit light of a green wavelength, a blue wavelength, a red wavelength, an infrared wavelength, or the like, but is not limited thereto. A plurality of detectors 112 may be disposed at different distances from the light source 111. In this case, the number, arrangement, and the like of the plurality of light sources 111 and the detector 112 may vary without limitation.

In addition, the spectrometer 110 may include a spectral filter, for example, a linear variable filter (LVF), but is not limited thereto. The LVF may have spectral properties that linearly vary across the entire length of the LVF. Thus, the LVF can disperse an incident light into a spectrum according to the order of wavelength. Though an LVF is compact in size, the LVF has powerful spectral capability.

The spectrometer 110 may measure a spectrum for calibration at the time of calibration. The spectrum for calibration may include a reference spectrum, and a spectrum for extracting a background signal, and the time of calibration may mean a user's fasting period, but is not limited thereto. For example, the spectrometer 110 may measure a reference spectrum in a stable state and/or a spectrum for extracting a background signal in a state other than the stable state. In this case, the stable state may mean a resting state in which a user's movement is not detected, but is not limited thereto.

The processor 120 may be electrically connected to the spectrometer 110. The processor 120 may control the spectrometer 110 according to the user's request, and may receive a spectrum measured by the spectrometer 110.

For example, the processor 120 may control the spectrometer 110 to measure a spectrum for calibration at the time of calibration. Here, the time of calibration may refer to a user's fasting period during which there is no significant change in blood glucose as described above, but is not limited thereto. For example, the processor 120 may control the spectrometer 110 to measure a reference spectrum in a stable state according to a user's request for calibration or according to a preset period, and/or may control the spectrometer 110 to measure a spectrum for extracting a background signal for a predetermined length of time and at a predetermined time interval.

The processor 120 may generate a personalized blood glucose estimation model based on the spectrum received from the spectrometer 110. In particular, the processor 120 may determine a calibration section in the measured spectrum, and may generate a personalized blood glucose estimation model based on a background signal extracted from the spectrum of the determined calibration section.

The processor 120 may determine a calibration section from the measured spectrum.

The processor 120 may determine the calibration section based on a change in a contact state between the object and the spectrometer 110. In particular, the processor 120 may determine that a section in which the change in the contact state between the object and the spectrometer 110 is greater than or equal to a predetermined reference is the calibration section.

For example, the processor 120 may compare the reference spectrum in the stable state measured by the spectrometer 110 with the spectrum for extracting a background signal in a state other than the stable state to monitor the change in the contact state, and may determine the calibration section. In this case, the stable state may mean a resting state in which a user's movement is not detected, but is not limited thereto. The processor 120 may compare the reference spectrum with the spectrum for extracting a background signal, may determine that a section in which a measured difference in the spectrum as a result of the comparison is greater than or equal to a predetermined reference is a section in which the change in the contact state is greater than or equal to the predetermined reference, and may determine that the determined section is a calibration section. However, the examples of the object are not limited thereto.

The processor 120 may induce a change in a contact state between the spectrometer 101 and the object.

For example, the processor 120 may induce the change in contact state, based on a monitoring result of the change in contact state. For example, when there is no section in which a change in a contact state is greater than or equal to a predetermined reference within a predetermined time from the start of the spectrum measurement or during the entire time of the spectrum measurement, the change in contact state may be induced.

In another example, in response to a received request for calibration, or according to a predetermined calibration period, the processor 120 may control the spectrometer 110 to induce the change in the contact state before or at the same time a spectrum for calibration is measured.

The processor 120 may guide the object to move in a predetermined direction, and/or may control the movement of the spectrometer 110 to induce the change in a contact state.

For example, the processor 120 may induce the change in contact state between the spectrometer 110 and the object by controlling the movement of the spectrometer 110.

In one example, the spectrometer 110 may further include a pressurizer which comes into contact with the object and presses the object to move in a predetermined direction. The processor 120 may control the pressurizer of the spectrometer 110 to move the object in a predetermined direction, and the pressurizer may include an elastic member, but is not limited thereto.

In another example, the spectrometer 110 may further include an actuator which moves a position of the spectrometer 110. The processor 120 may control the actuator of the spectrometer 110 to move the spectrometer 110, for example, in the left and right directions.

The processor 120 may extract a background signal from a spectrum of the determined calibration section. In particular, the background signal may include, but not limited to, at least one of a water signal, a keratin signal, an elastic signal, a collagen signal, or a fat signal, or a combination thereof.

For example, the processor 120 may extract the background signal by performing at least one of differential, principal component analysis, independent component analysis, non-negative matrix factorization, or auto-encoding with respect to the spectrum for extracting a background signal in the determined calibration section. However, the present disclosure is not limited thereto, such that the processor 120 may extract the background signal using various background signal extraction methods.

The processor 120 may verify whether the extracted background signal is valid.

For example, the processor 120 may compare the extracted background signal with at least one of a reference signal or a user's existing background signal present in a user-specific personalized background signal database and may verify whether the extracted background signal is valid, based on a comparison result. Here, the reference signal may refer to a pure spectrum of a material that corresponds to the extracted background signal. For example, in a case where the extracted background signal is a water signal, the reference signal may refer to a water spectrum. The user's existing background signal may refer to a plurality of background signals of the corresponding user which are extracted as a result of a plurality of calibrations.

For example, the processor 120 may calculate a similarity and/or correlation between the extracted background signal and the reference signal, and verify whether the extracted background signal is valid based on the calculated similarity and/or correlation. The processor 120 may determine that the extracted background signal is invalid when a statistical value (e.g., average, maximum, minimum, variance, standard deviation, etc.) of the calculated similarity and/or correlation is less than a predetermined reference value. At this time, the processor 120 may determine, based on a variation in the calculated similarity and/or correlation, whether the extracted background signal abruptly changes as the measurement time has elapsed. When it is determined that the extracted background signal abruptly changes, the processor 120 may determine that the extracted background signal is invalid. At this time, the similarity may be calculated using a similarity calculation algorithm including Euclidean distance, Manhattan distance, cosine distance, Mahalanobis distance, a Jaccard coefficient, an extended Jaccard coefficient, a Pearson's correlation coefficient, and a Spearman's correlation coefficient.

In another example, the processor 120 may verify whether the extracted background signal is valid by using a method of analyzing noise or change pattern of the extracted background signal. For example, the processor 120 may calculate noise of the extracted background signal. When the noise exceeds a predetermined reference value, the processor 120 may determine that the extracted background signal is valid. Alternatively, the processor 120 may perform fast Fourier transform (FFT) to convert the extracted background signal into a signal of a frequency domain. When low-frequency random noise is detected in a signal converted into a frequency domain, the processor 120 may determine that the extracted background signal is invalid.

When it is determined that the extracted background signal is invalid as a result of verification of the validity of the extracted background signal, the processor 120 may re-determine a calibration section from the measured spectrum for extracting a background signal or re-extract a background signal from the determined calibration section.

Alternatively, when it is determined that the extracted background signal is invalid as a result of verification of the validity of the extracted background signal, the processor 120 may guide the user to re-measure a spectrum. In particular, the processor 120 may generate information to induce the user to a change in contact state between the object and the spectrometer 110. For example, the processor 120 may guide the user to move the object in a predetermined direction, or may control the movement of the spectrometer 110 to induce the change in contact state between the spectrometer 110 and the object.

The processor 120 may perform preprocessing on the extracted background signal. The preprocessing may include at least one of baseline correction, scattering correction, normalization, and filtering, but is not limited thereto.

The processor 120 may generate a personalized blood glucose estimation model based on the extracted background signal. In particular, the processor 120 may generate the personalized blood glucose estimation model using Lambert-Beer's law as shown in Equation 1 below, but is not limited thereto.

S=ε_{b1}LC_{b1}+ε_{b2}LC_{b2}+ε_{b3}LC_{b3}+ε_{b4}LC_{b4}+ε_{g}LC_{g} (1)

Here, S denotes a spectrum for estimating blood glucose, which is measured at the time of actual blood glucose estimation, ε_{b1}, ε_{b2}, ε_{b3}, and ε_{b4} denote the unique light absorption of background signals b₁, b₂, b₃, and b₄, respectively, and ε_{g} denotes the unique light absorption of blood glucose. L denotes a light path, C_{b1}, C_{b2}, C_{b3}, and C_{b4} denote the concentration of each of the background signals b₁, b₂, b₃, and b₄, respectively, and C_{g} denotes a blood glucose concentration.

The processor 120 may estimate blood glucose of the user based on the generated personalized blood glucose estimation model and the spectrum for estimating blood glucose, which is measured by the spectrometer 110.

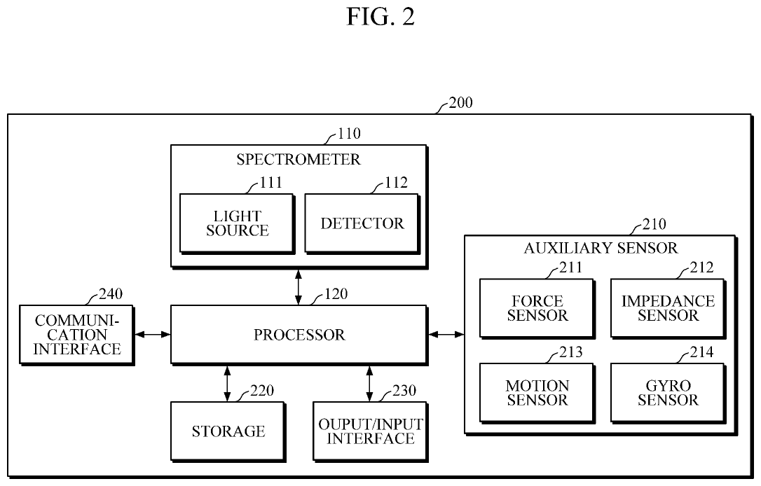

FIG. 2 is a block diagram illustrating an apparatus 200 for estimating blood glucose according to another exemplary embodiment. Referring to FIG. 2, the apparatus 200 for estimating blood glucose may include a spectrometer 110, a processor 120, an auxiliary sensor 210, a storage 220, an output and input interface 230, and a communication interface 240. The spectrometer 110 and a light source 111 and a detector 112 included in the spectrometer 110 are described in detail with reference to FIG. 1, and hence, hereinafter, the processor 120, the auxiliary sensor 210, the storage 220, the output and input interface 230, and the communication interface 240 will be described in detail.

The processor 120 may monitor a contact state between an object and the spectrometer 110 and determine a calibration section based on the monitored change in the contact state.

In one example, the processor 120 may monitor a change in the contact state between the object and the spectrometer 110 based on measurement data of the auxiliary sensor 210 and determine a calibration section. In particular, the auxiliary sensor 210 may include a force sensor 211, an impedance sensor 212, a motion sensor 213, and a gyro sensor 214, but is not limited thereto.

For example, the processor 120 may determine that a force between the object and the spectrometer 110, which is measured by the force sensor 211, is the contact state, and may determine that a section in which a change in the measured force is greater than or equal to a predetermined reference is a calibration section. The force sensor 211 may include a force sensor array. The processor 120 may determine that a section in which the distribution of the force at each position measured by the force sensor array is changed to a predetermined reference or higher is a calibration section. However, the present disclosure is not limited thereto, such that the processor 120 may determine that a section in which a movement of the user measured by the impedance sensor 212, the motion sensor 213, and/or the gyro sensor 214 is greater than or equal to a predetermined reference is a calibration section.

In another example, as described above with reference to FIG. 1, the processor 120 may compare a reference spectrum measured in a stable state by the spectrometer 110 with a spectrum for extracting a background signal measured in a state other than the stable state to monitor a change in a contact state, and may determine a calibration section. A detailed description thereof will be omitted.

In another example, the processor 120 may determine a calibration section based on a result of comparing the reference spectrum and the spectrum for extracting a background signal, along with the measurement data of the auxiliary sensor 210.

The processor 120 may guide the object to move in a predetermined direction, and/or may control the movement of the spectrometer 110 to induce the change in a contact state.

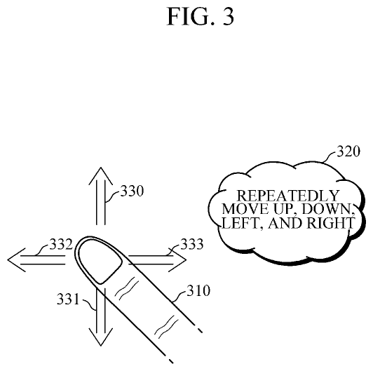

In one example, the processor 120 may control the output and input interface 230 to display a graphic object that guides the user to move the object in a predetermined direction and guide a change in the contact state between the spectrometer 110 and the object. An example of the graphic object displayed by the output and input interface 230 through a display module under the control of the processor 120 will be described with reference to FIG. 3.

FIG. 3 illustrates an example of a graphic object guiding an object to move in a predetermined direction. Referring to FIG. 3, a graphic object guiding the object to move in a predetermined direction may include a graphic object 310 representing the shape of the object, a text graphic object 320, such as “Repeatedly move up, down, left, and right,” and figure graphic objects 330, 331, 332, and 333 such as arrows. The figure graphic objects 330, 331, 332, and 333 may be sequentially displayed according to an order of directions in which the object 310 is to move, but the present disclosure is not limited thereto. FIG. 3 illustrates an object 310 as a finger, without being limited thereto, and the object may be another body part, such as an upper area of a wrist, as described above. Also, FIG. 3 illustrates an example in which the movement of the object is visually guided through a display module, without being limited thereto, and the output and input interface 230 may guide the movement of the object in a non-visual manner through vibration, tactile sensation, and the like by using a speaker module or a haptic module.

In another example, as described above with reference to FIG. 1, the processor 120 may control the movement of the spectrometer 110 to induce a change in the contact state between the spectrometer 110 and the object. A detailed description thereof will be omitted.

The storage 220 may store a processing result of the spectrometer 110 and/or a processing result of the processor 120. Also, the storage 220 may store a reference signal, a user-specific personalized background signal database, and extracted background signals. The storage 220 may store various types of reference information that may be necessary for blood glucose estimation. For example, the reference information may include user characteristic information, such as the user's age, gender, health status, and the like, that is received through the communication interface 240 and/or the output and input interface 230. In addition, the reference information may include information, such as a blood glucose estimation model, criteria for blood glucose estimation, a calibration period, a reference force set for each user, and/or a reference distribution of force. However, the examples of the reference information are not limited thereto.

In particular, the storage 220 may include at least one storage medium of a flash memory type memory, a hard disk type memory, a multimedia card micro type memory, a card type memory (e.g., an SD memory, an XD memory, etc.), a random access memory (RAM), a static random access memory (SRAM), a read only memory (ROM), an electrically erasable programmable read only memory (EEPROM), a programmable read only memory (PROM), a magnetic memory, a magnetic disk, and an optical disk, and the like, but is not limited thereto.

The output and input interface 230 may output guide information for a contact state change generated by the processor 120 as described above. For example, the output and input interface 230 may visually output graphic objects guiding the object in a predetermined direction through the display module as shown in FIG. 3, or may output guide information in a non-visual manner through vibration, tactile sensation, and the like by using a speaker module or a haptic module.

When it is determined that an extracted background signal is invalid as a result of verification of the validity of the extracted background signal, the output and input interface 230 may guide the user to re-measure a spectrum by using a visual or non-visual method under the control of the processor 120. In particular, the output and input interface 230 may also output guide information for a contact state change, which is generated by the processor 120.

The output and input interface 230 may output a spectrum measured by the spectrometer 110, a blood glucose value estimated by the processor 120, and/or guide information for the estimated blood glucose value. For example, the output and input interface 230 may visually output data processed by the spectrometer 110 or the processor 120, or output the data in a non-visual manner through voice, vibration, tactile sensation, and the like by using a speaker module or a haptic module. A display area may be divided into two or more areas, in which the measured spectrum, the distribution of contact force, and the like may be output in various forms of graphs in a first area; and an estimated blood glucose value may be output in a second area. In particular, if an estimated blood glucose value falls outside a normal range, the output and input interface 230 may output warning information in various manners, such as highlighting an abnormal value in red and the like, displaying the abnormal value along with a normal range, outputting a voice warning message, adjusting a vibration intensity, and the like.

The communication interface 240 may communicate with an external device by using wired or wireless communication techniques under the control of the processor 120, and may transmit and receive various data to and from the external device. For example, the communication interface 240 may transmit an estimation result of blood glucose to the external device, and may receive various types of reference information (e.g., a user's age, gender, health status, etc.) for estimating blood glucose from the external device. Examples of the external device may include an invasive blood sugar measuring device, and an information processing device such as a smartphone, a tablet PC, a desktop computer, a notebook computer, and the like.

Examples of the communication techniques may include Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, Radio Frequency Identification (RFID) communication, 3G communication, 4G communication, 5G communication, and the like. However, this is an example and is not intended to be limiting.

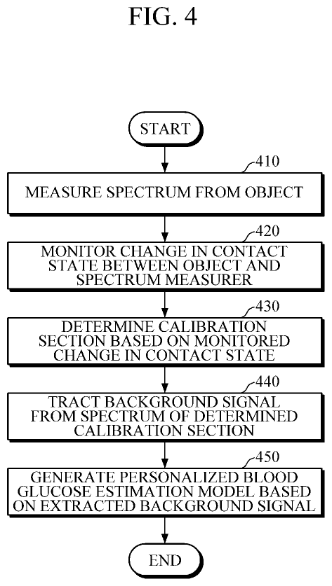

FIG. 4 is a flowchart illustrating a method of estimating blood glucose according to an exemplary embodiment. The method of FIG. 4 is one exemplary embodiment of a method performed by the apparatuses for estimating blood glucose of FIGS. 1 and 2, which is described above, and thus will be described briefly to avoid redundancy.

First, a spectrum may be measured from an object in operation 410. The spectrum may be measured during a user's fasting period, but the present disclosure is not limited thereto.

Then, a change in the contact state between the object and the spectrometer may be monitored in operation 420. At this time, the change in the contact state may be monitored based on measurement data of an auxiliary sensor, or the change in the contact state may be monitored by comparing a reference spectrum measured in a stable state with a spectrum for extracting a background signal in a state other than the stable state.

Then, a calibration section may be determined based on the monitored change in the contact state in operation 430. At this time, a section in which the monitored change in the contact state is greater than or equal to a predetermined reference may be determined to be the calibration section. In one example, a force measured by a force sensor may be determined as a contact state and a section in which a change in the measured force is greater than or equal to a predetermined reference may be determined to be a calibration section. In another example, a reference spectrum and a spectrum for extracting a background signal may be compared, and a section in which a measured difference in the spectrum as a result of the comparison is greater than or equal to a predetermined reference may be determined to be a section in which the change in the contact state is greater than or equal to the predetermined reference, and may be determined to be a calibration section.

At this time, if there is no section in which the monitored change in the contact state is greater than or equal to a predetermined reference, a change in the contact state may be induced.

Then, a background signal may be extracted from a spectrum of the determined calibration section in operation 440. In particular, the background signal may include, but not limited to, at least one of a water signal, a keratin signal, an elastic signal, a collagen signal, or a fat signal, or a combination thereof. For example, the background signal may be extracted by performing at least one of differential, principal component analysis, independent component analysis, non-negative matrix factorization, or auto-encoding for the spectrum for extracting a background signal in the determined calibration section. However, the present disclosure is not limited thereto.

Then, a personalized blood glucose estimation model may be generated based on the extracted background signal in operation 450. At this time, Lambert-Beer's law may be used, but the present disclosure is not limited thereto. In addition, pre-processing including at least one of baseline correction, scattering correction, normalization, and filtering may be performed on the extracted background signal.

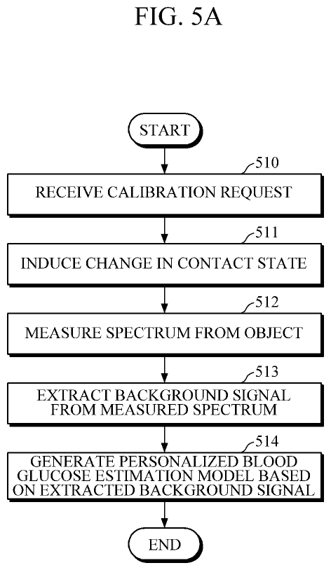

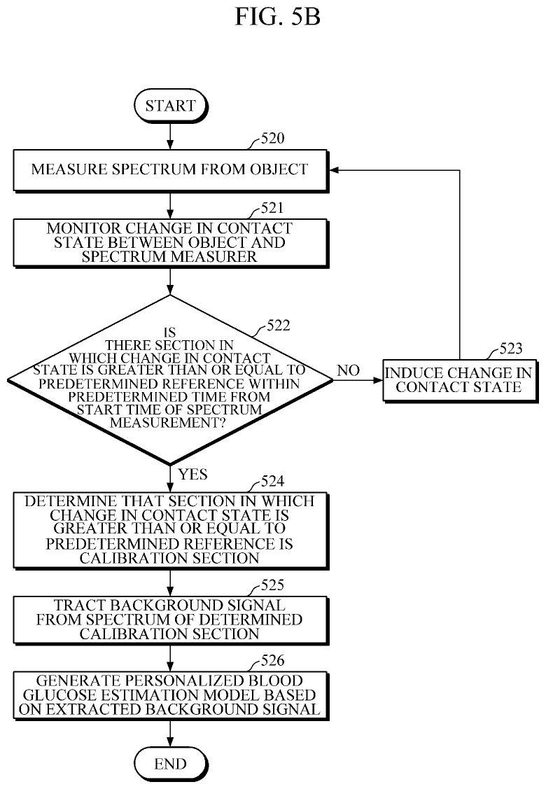

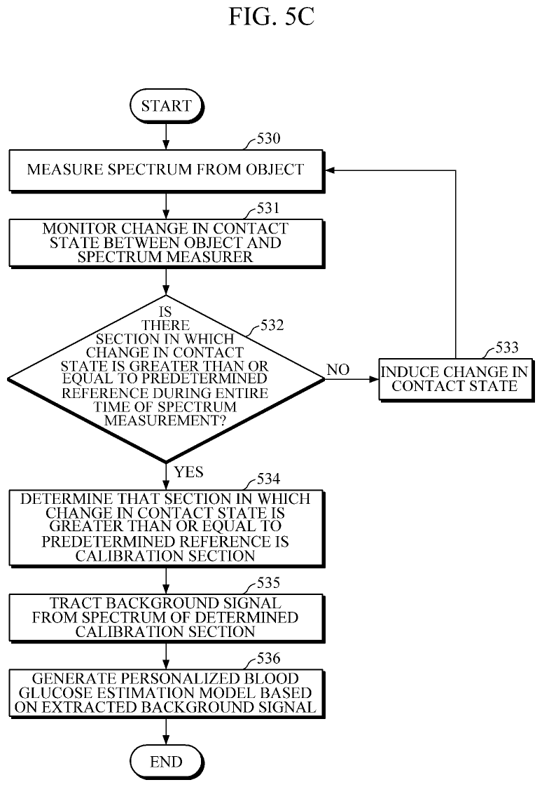

FIGS. 5A to 5C are flowcharts illustrating a method of estimating blood glucose according to another exemplary embodiment. The method of FIGS. 5A to 5C is an exemplary embodiment of the method performed by the apparatuses for estimating blood glucose of FIGS. 1 and 2, which is described above, and thus will be described briefly to avoid redundancy.

FIG. 5A is a flowchart illustrating a process of performing calibration by inducing a change in a contact state before measuring a spectrum.

Referring to FIG. 5A, a calibration request may be received from a user in operation 510. However, operation 510 may be omitted, and instead, the following operations 511 to 514 may be performed according to a predetermined period.

Then, a change in a contact state may be induced in operation 511. At this time, an object may be guided to move in a predetermined direction, and/or the movement of the spectrometer may be controlled to induce the change in the contact state.

Next, a spectrum may be measured from the subject in operation 512.

Then, a background signal may be extracted from the measured spectrum in operation 513.

Then, a personalized blood glucose estimation model may be generated based on the extracted background signal in operation 514.

FIG. 5B is a flowchart illustrating a process of performing calibration by inducing a change in a contact state when there is no section in which the change in the contact state is greater than or equal to a predetermined reference within a predetermined time from the start of spectrum measurement.

Referring to FIG. 5B, first, a spectrum may be measured from an object in operation 520.

Then, a change in the contact state between the object and the spectrometer may be monitored in operation 521.

Next, it may be determined whether there is a section in which the change in the contact state is greater than or equal to a predetermined reference within a predetermined time from the start of the spectrum measurement in operation 522.

When it is determined that there is no such section, a change in the contact state may be induced in operation 523. At this time, an object may be guided to move in a predetermined direction, and/or the movement of the spectrometer may be controlled to induce the change in the contact state.

When it is determined that such a section is present, the section in which the change in the contact state is greater than or equal to the predetermined reference may be determined to be a calibration section in operation 524.

Then, a background signal may be extracted from the spectrum of the determined calibration section in operation 525.

Then, a personalized blood glucose estimation model may be generated based on the extracted background signal in operation 526.

FIG. 5C is a flowchart illustrating a process of performing calibration by inducing a change in a contact state when there is no section in which the change in the contact state is greater than or equal to a predetermined reference during the entire time of spectrum measurement.

Referring to FIG. 5C, first, a spectrum may be measured from an object in operation 530.

Then, a change in the contact state between the object and the spectrometer may be monitored in operation 531.

Next, it may be determined whether there is a section in which the change in the contact state is greater than or equal to a predetermined reference during the entire time of the spectrum measurement in operation 532.

When it is determined that there is no such section, a change in the contact state may be induced in operation 533. At this time, an object may be guided to move in a predetermined direction, and/or the movement of the spectrometer may be controlled to induce the change in the contact state.

When it is determined that such a section is present, the section in which the change in the contact state is greater than or equal to the predetermined reference may be determined to be a calibration section in operation 534.

Then, a background signal may be extracted from the spectrum of the determined calibration section in operation 535.

Then, a personalized blood glucose estimation model may be generated based on the extracted background signal in operation 536.

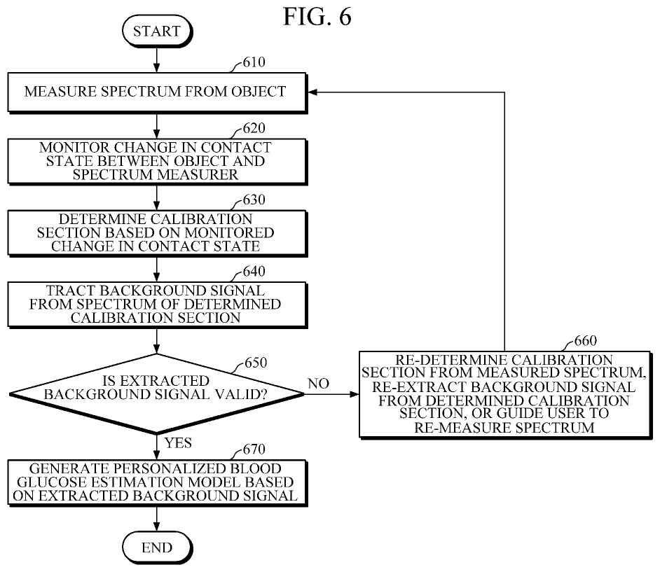

FIG. 6 is a flowchart illustrating a method of estimating blood glucose according to still another exemplary embodiment. The method of FIG. 6 is one exemplary embodiment of the method performed by the apparatuses for estimating blood glucose of FIGS. 1 and 2, which is described above, and thus will be described briefly to avoid redundancy.

First, a spectrum may be measured from an object in operation 610. The spectrum may include a spectrum for extracting a background signal.

Then, a change in the contact state between the object and the spectrometer may be monitored in operation 620.

Then, a calibration section may be determined based on the monitored change in the contact state in operation 630.

Then, a background signal may be extracted from a spectrum of the determined calibration section in operation 640.

Next, it may be determined whether the extracted background signal is valid in 650.

In one example, the extracted background signal may be compared with at least one of a reference signal or a user's existing background signal present in a user-specific personalized background signal database to calculate a similarity or correlation therebetween. Then, it may be determined whether the calculated similarity or correlation is greater than or equal to a predetermined reference value.

In particular, the extracted background signal may be determined to be invalid when a statistical value (e.g., average, maximum, minimum, variance, standard deviation, etc.) of the calculated similarity and/or correlation is less than the predetermined reference value. At this time, based on a variation in the calculated similarity and/or correlation, whether the extracted background signal abruptly changes as the measurement time has elapsed may be determined. When it is determined that the extracted background signal abruptly changes, the extracted background signal may be determined to be invalid. At this time, the similarity may be calculated using a similarity calculation algorithm including Euclidean distance, Manhattan distance, cosine distance, Mahalanobis distance, a Jaccard coefficient, an extended Jaccard coefficient, a Pearson's correlation coefficient, and a Spearman's correlation coefficient.

In another example, whether the extracted background signal is valid may be verified by using a method of analyzing noise or change pattern of the extracted background signal. A detailed description thereof will be omitted.

When it is determined that the extracted background signal is invalid, a calibration section may be re-determined from the measured spectrum, a background signal may be re-extracted from the determined calibration section, or the user may be guided to re-measure a spectrum in 660.

When it is determined that the extracted background signal is valid, a personalized blood glucose estimation model may be generated based on the extracted background signal in 670.

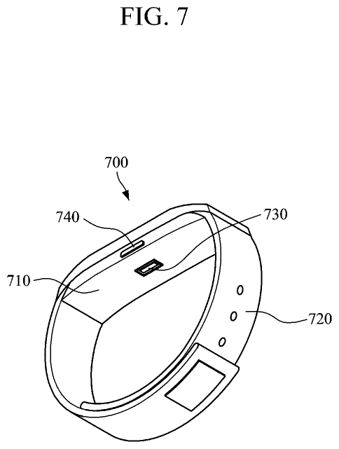

FIG. 7 illustrates a wearable device according to an exemplary embodiment. A wearable device 700 may include various exemplary embodiments of the above-described apparatuses 100 and 200 for estimating blood glucose.

Referring to FIG. 7, the wearable device 700 may include a main body 720 and a strap 720.

The strap 720 may be connected to both ends of the main body 700 and be made of a flexible material to conform to a user's wrist. The strap 720 may include a first strap and a second strap that are separate from each other. One ends of the first strap and the second strap may be connected to each end of the main body 710 and may be fastened to each other using fastening means formed on the other sides thereof. In this case, the fastening means may be formed as a Velcro fastening means, a pin fastening means, a pin fastening means, or the like, but is not limited thereto. In addition, the strap 720 may be formed as one integrated piece, such as a band, which is not separated into pieces.

In this case, air may be injected into the strap 820 or an airbag may be included in the strap 720, so that the strap 720 may have elasticity according to a change in pressure applied to the wrist, and the change in pressure of the wrist may be transmitted to the main body 710.

A battery, which supplies power to the wearable device 700, may be embedded in the main body 710 or the strap 720.

Further, a sensor part 730 may be mounted on one surface of the main body 710. The sensor part 730 may include a spectrum measure and an auxiliary sensor including a force sensor, an impedance sensor, a motion sensor, a gyro sensor, and the like. In this case, the spectrometer may include a light source and a CIS-based image sensor.

The processor may be mounted in the main body 710. The processor may generate a personalized blood glucose estimation model based on the spectrum measured by the sensor part 730. For example, the processor may determine a calibration section based on a change in a contact state between an object and the spectrometer included in the sensor part 730, extract a background signal from a spectrum of the determined calibration section, and generate a personalized blood glucose estimation model based on the extracted background signal. A detailed description thereof will be omitted.

The processor may display a graphic object guiding the object to move in a predetermined direction through an output and input interface when a calibration request is received from the user. When blood glucose is estimated, the processor may provide the estimation result to the user through the output and input interface. The output and input interface may be mounted on a front surface of the main body 710 and may output guide information and/or a blood glucose estimation result.

A storage may be included in the main body 710 and may store information processed by the processor and reference information necessary for blood glucose estimation, for example, a reference signal, a user-specific personalized background signal database, and the like.

In addition, the wearable device 700 may include a manipulator 740 configured to receive a user's control command and transmit it to the processor. The manipulator 740 may be mounted on one side of the main body 710, and may have a function for inputting a command to turn on/off the wearable device 700.

Additionally, the wearable device 700 may include a communication interface configured to transmit and receive various types of data to and from an external device, and various modules configured to perform additional functions provided by the wearable device 700.

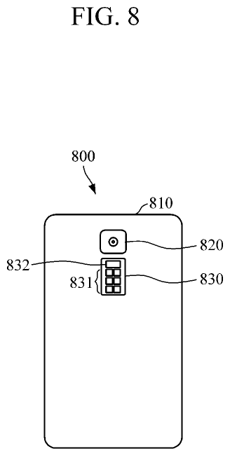

FIG. 8 illustrates a smart device according to an exemplary embodiment. A smart device 800 may include various exemplary embodiments of the above-described apparatuses 100 and 200 for estimating blood glucose. Here, the smart device may include a smartphone, a tablet PC, and the like.

Referring to FIG. 8, the smart device 800 may include a main body 810 and a sensor part 830 disposed on one surface of the main body 910. The sensor part 930 may include at least one light source 831 and at least one detector 832 and may measure a spectrum from an object. In particular, the detector 832 may include a contact image sensor (CIS)-based image sensor. The sensor part 830 may be disposed on the rear surface of the main body 810 as illustrated, but is not limited thereto. Also, the sensor part 830 may include an auxiliary sensor, such as an impedance sensor, a motion sensor, a gyro sensor, or the like.

The processor is mounted on the main body 810 and may generate a personalized blood glucose estimation model based on the spectrum measured by the sensor part 830.

Meanwhile, an image sensor 820 may be mounted in the main body 810 as illustrated, and the image sensor 820 may capture an image of an object, for example, a finger, when the user approaches the finger to the sensor part 830 to measure a spectrum and may transmit the image to the processor. In this case, the processor may identify a relative position of the finger relative to the actual position of the sensor part 830 and provide, through an output and input interface, the user with the relative position of the finger and a graphic object guiding the object to move in a predetermined direction in order to induce change in a contact state.

In addition, a storage, a communication interface, and the like may be mounted in the main body 810, whereby a blood glucose estimated by the processor 810 may be stored or may be transmitted to other external devices. In addition, various modules configured to perform various functions may be mounted in the main body 810.

While not restricted thereto, an example embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an example embodiment may be written as a computer program transmitted over a computer-readable transmission medium, such as a carrier wave, and received and implemented in general-use or special-purpose digital computers that execute the programs. Moreover, it is understood that in example embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for estimating blood glucose, the apparatus comprising:
a spectrometer configured to measure a spectrum from an object; and
a processor configured to monitor a change in a contact state between the object and the spectrometer, determine a calibration section based on the monitored change in the contact state, extract a background signal from the spectrum of the determined calibration section, and generate a personalized blood glucose estimation model based on the extracted background signal.

2. The apparatus of claim 1, wherein the spectrometer comprises a light source configured to emit light to the object and a detector configured to detect a light signal by receiving the light scattered or reflected from the object.

3. The apparatus of claim 1, further comprising: at least one of a force sensor, an impedance sensor, a motion sensor, or a gyro sensor,
wherein the processor is further configured to monitor the change in the contact state between the object and the spectrometer based on measurement data of the sensor.

4. The apparatus of claim 1, wherein the processor is further configured to compare a reference spectrum in a stable state with the spectrum measured by the spectrometer to monitor the change in the contact state between the object and the spectrometer.

5. The apparatus of claim 1, wherein the processor is further configured to determine that a section in which the monitored change in the contact state is greater than or equal to a predetermined reference is the calibration section.

6. The apparatus of claim 1, wherein the processor is further configured to induce the change in the contact state when a calibration request is received, or when there is no section in which the change in the contact state is greater than or equal to a predetermined reference within a predetermined time from a start of a spectrum measurement, or during an entire time of the spectrum measurement.

7. The apparatus of claim 6, wherein the processor is further configured to induce the change in the contact state by controlling a movement of the spectrometer.

8. The apparatus of claim 6, further comprising a display configured to display a graphic object guiding the object to move in a predetermined direction.

9. The apparatus of claim 1, wherein the processor is further configured to extract the background signal, based on the spectrum of the determined calibration section, by using at least one of differential, principal component analysis, independent component analysis, non-negative matrix factorization, or auto-encoding.

10. The apparatus of claim 1, wherein the processor is further configured to compare the extracted background signal with at least one of a reference signal or a user's existing background signal present in a user-specific personalized background signal database to calculate a similarity or correlation therebetween, and verify whether the extracted background signal is valid, based on the calculated similarity or correlation.

11. The apparatus of claim 10, wherein, when the extracted background signal is determined to be invalid, the processor is further configured to re-determine a calibration section from the measured spectrum, extract another background signal from the spectrum of the determined calibration section, or guide the spectrometer to obtain another spectrum.

12. The apparatus of claim 1, wherein the processor is further configured to perform pre-processing including at least one of baseline correction, scattering correction, normalization, or filtering on the extracted background signal.

13. The apparatus of claim 1, wherein the spectrometer is further configured to measure the spectrum from the object at a time of estimating blood glucose and the processor is further configured to estimate the blood glucose based on the spectrum measured at the time of estimating blood glucose and the personalized blood glucose estimation model.

14. The apparatus of claim 1, further comprising a storage configured to comprise a reference signal and a user-specific personalized background signal database,
wherein the storage is configured to store the extracted background signal in the personalized background signal database.

15. A method of estimating blood glucose, comprising:
measuring a spectrum from an object;
monitoring a change in a contact state between the object and a spectrometer;
determining a calibration section based on the monitored change in the contact state;
extracting a background signal from the spectrum of the determined calibration section; and
generating a personalized blood glucose estimation model based on the extracted background signal.

16. The method of claim 15, wherein the determining of the calibration section comprises determining that a section in which the monitored change in the contact state is greater than or equal to a predetermined reference is the calibration section.

17. The method of claim 15, further comprising inducing the change in the contact state when a calibration request is received, or when there is no section in which the change in the contact state is greater than or equal to a predetermined reference within a predetermined time from a start of a spectrum measurement, or during an entire time of the spectrum measurement.

18. The method of claim 15, further comprising comparing the extracted background signal with at least one of a reference signal or a user's existing background signal present in a user-specific personalized background signal database to calculate a similarity or correlation therebetween, and verifying whether the extracted background signal is valid, based on the calculated similarity or correlation.

19. A wearable device comprising:
an optical sensor comprising a plurality of light emitting diodes (LEDs) configured to emit at least a green light, a red light, an infrared light to a user, and a plurality of photodiodes configured to measure light reflected back from the user;
a motion sensor configured to obtain a motion signal by measuring a motion of the user while the wearable device is worn by the user; and
a processor configured to:
obtain personal information of the user, the personal information comprising information of a gender and an age of the user;
during a calibration stage, control the optical sensor to obtain a reference biological signal while the user in a resting state and the wearable device is in contact with the user; and
during an estimation stage, obtain a biological signal based on the light measured by the plurality of photodiode detectors, correct the biological signal based on the motion signal, the personal information, and the reference signal, and obtain health information of the user based on the corrected biological signal.

20. The wearable device of claim 19, wherein the processor is further configured to:
extract a background signal from the reference biological signal that is obtained during the calibration stage; and
correct the biological signal based the background signal of the reference signal.
